(19) United States
(12) Patent Application Publication
(10) Pub. No.: US 2019/0287673 A1
(43) Pub. Date: Sep. 19, 2019

(54) MEDICAL IMAGE PROCESSING DEVICE, MEDICAL OBSERVATION APPARATUS, AND MEDICAL OBSERVATION APPARATUS OPERATION METHOD

(71) Applicant: Sony Olympus Medical Solutions Inc., Tokyo (JP)

(72) Inventors: Taihei Michihata, Kanagawa (JP); Yuichi Yamada, Tokyo (JP)

(73) Assignee: Sony Olympus Medical Solutions Inc., Tokyo (JP)

(21) Appl. No.: 16/259,046

(22) Filed: Jan. 28, 2019

(30) Foreign Application Priority Data
Mar. 13, 2018 (JP) 2018-045951

(51) Int. Cl. G16H 30/40; G06T 7/00; A61B 1/00; A61B 1/05

(52) U.S. Cl. CPC G16H 30/40; G06T 7/0012; G06T 2207/30096; A61B 1/05; A61B 1/00009

(57) ABSTRACT

A disclosed medical image processing device includes a motion amount calculation unit which compares a first image captured of a subject with a second image captured of the subject, the second image having been captured temporally in advance with respect to the first image, and calculates a motion amount from the second image for each area in the first image; an area partitioning unit which partitions the entire area of the first image into a first area in which the motion amount is within a predetermined range and a second area in which the motion amount is out of the predetermined range; and an identification image generation unit which generates an identification image in which the first area and the second area of the first image are identified.

### CROSS-REFERENCE TO RELATED APPLICATION(S)

The present application claims priority to and incorporates by reference the entire contents of Japanese Patent Application No. 2018-045951 filed with Japan Patent Office on Mar. 13, 2018.

### BACKGROUND

The present disclosure relates to a medical image processing device, a medical observation apparatus, and a medical observation apparatus operation method. In the past, a medical observation apparatus for observing the inside of a subject (the inside of a living body) of a person or the like is known in a medical field (for example, see Japanese Laid-open Patent Publication No. 2015-134039 A, referred to as JP 2015-134039 A hereinafter).

The medical observation apparatus (endoscope apparatus) described in JP 2015-134039 A includes an insertion unit which is inserted into a subject and acquires a subject image inside the subject from a distal end thereof, a camera head which is detachably connected to an eyepiece of the insertion unit and captures an image of the subject to generate a captured image, a control device which processes the captured image to generate a video signal for display, and a display device which displays the captured image based on the video signal for display.

### SUMMARY

In a living body, a site such as a tumor is harder than other sites. Then, for example, when the site such as a tumor exists in a beating heart or the like, the site such as a tumor cannot easily move with respect to the other sites since the site is harder than the other sites. For this reason, when there is a function of generating an identification image in which a large motion area and a small motion area of the captured image can be identified, a doctor or the like can easily find and diagnose the tumor or the like by checking the identification image. That is, it is possible to improve convenience.

However, since the above-described function is not provided in the medical observation apparatus described in JP 2015-134039 A, it is difficult to improve convenience.

The present disclosure, which has been made in view of the above-described circumstances, is directed to a medical image processing device, a medical observation apparatus, and a medical observation apparatus operation method capable of improving convenience.

According to a first aspect of the present disclosure, a medical image processing device is provided which includes a motion amount calculation unit which compares a first image captured of a subject with a second image captured of the subject, the second image having been captured temporally in advance with respect to the first image, and calculates a motion amount from the second image for each area in the first image; an area partitioning unit which partitions the entire area of the first image into a first area in which the motion amount is within a predetermined range and a second area in which the motion amount is out of the predetermined range; and an identification image generation unit which generates an identification image in which the first area and the second area of the first image are identified.

According to a second aspect of the present disclosure, a medical image processing device is provided which includes an immovable area calculation unit which compares a first image captured of a subject with a second image captured of the subject, the second image having been captured temporally in advance with respect to the first image, at corresponding pixels, and calculates an immovable area in which no motion from the second image occurs in the first image; and an identification image generation unit which generates an identification image in which the immovable area and the other areas of the first image are identified.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a medical observation apparatus according to a first embodiment;

FIG. 2 is a block diagram illustrating a camera head and a control device;

FIG. 3 is a diagram illustrating an example of a motion amount calculation process;

FIG. 4 is a diagram illustrating an example of the motion amount calculation process;

FIG. 5 is a diagram illustrating an example of the motion amount calculation process;

FIG. 6 is a diagram illustrating an example of an identification image generation process;

FIG. 7 is a flowchart illustrating a medical observation apparatus operation method;

FIG. 8 is a block diagram illustrating a medical observation apparatus according to a second embodiment;

FIG. 9 is a flowchart illustrating a medical observation apparatus operation method;

FIG. 10 is a block diagram illustrating a medical observation apparatus according to a third embodiment; and

FIG. 11 is a flowchart illustrating a medical observation apparatus operation method.

### DETAILED DESCRIPTION OF THE PREFERRED EMBODIMENTS

Hereinafter, a mode for carrying out the present disclosure (hereinafter, embodiments) will be described with reference to the drawings. Furthermore, the disclosure is not limited to the embodiments to be described below. Additionally, in the description of the drawings, the same reference numerals are given to the same parts.

### First Embodiment

Schematic Configuration of Medical Observation Apparatus

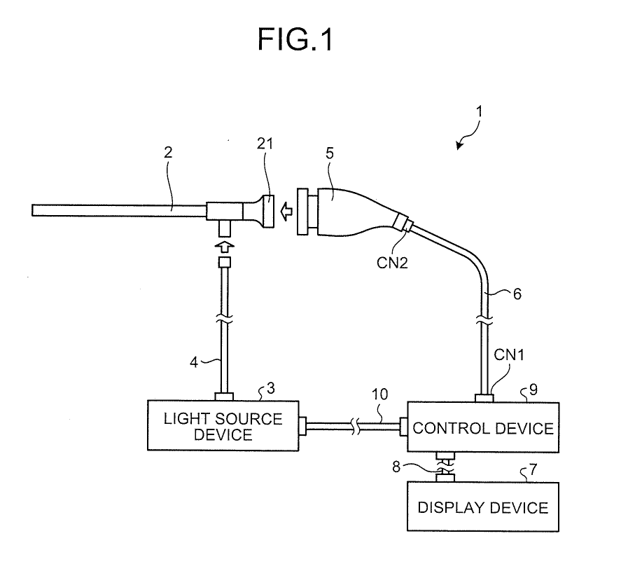

FIG. 1 is a diagram illustrating a medical observation apparatus according to a first embodiment.

A medical observation apparatus 1 is an apparatus which is used in a medical field and observes the inside of a living body. The medical observation apparatus 1 includes an insertion unit 2, a light source device 3, a light guide 4, a camera head 5, a first transmission cable 6, a display device 7, a second transmission cable 8, a control device 9, and a third transmission cable 10.

In the first embodiment, the insertion unit 2 is configured as a rigid endoscope. That is, the insertion unit 2 is of an elongated shape, entirely rigid, and inserted into a living body. Incidentally, a part of the insertion unit 2 may be flexible and the other part thereof may be rigid in other embodiments. The insertion unit 2 includes one or a plurality of lenses that constitute an optical system to condense a subject image.

The light source device 3, to which one end of the light guide 4 is connected, supplies light for illuminating the inside of the living body to the one end of the light guide 4 under the control of the control device 9. Although the light source device 3 is provided separately from the control device 9 in the first embodiment, a control device may be used which a light source device is incorporated therein.

While the one end of the light guide 4 is detachably connected to the light source device 3, the other end thereof is detachably connected to the insertion unit 2. With this, the light guide 4 receives the light at the one end from the light source device 3, and transmits the light therethrough to the other end and thus to the insertion unit 2. The light supplied to the insertion unit 2 is emitted from a distal end of the insertion unit 2 and thus illuminates the living body. The light which has been emitted to the living body and is reflected from the living body is condensed (as a subject image) by the optical system inside the insertion unit 2.

The camera head 5 corresponds to an imaging device according to this disclosure. The camera head 5 is detachably connected to a proximal end (an eyepiece 21 (FIG. 1)) of the insertion unit 2. Then, the camera head 5 captures the subject image condensed by the insertion unit 2 and outputs an image signal (RAW signal) due to the capturing under the control of the control device 9. The image signal is, for example, an image signal of 4K or more.

Furthermore, a detailed configuration of the camera head 5 will be described below.

One end of the first transmission cable 6 is detachably connected to the control device 9 through a connector CN1 and the other end thereof is detachably connected to the camera head 5 through a connector CN2. Then, the first transmission cable 6 transmits the image signal or the like output from the camera head 5 to the control device 9 and transmits each of a control signal, a synchronization signal, a clock signal, and electric power output from the control device 9 to the camera head 5.

Incidentally, the image signal or the like may be transmitted optically (or as an optical signal) or electrically (or as an electric signal) from the camera head 5 to the control device 9 through the first transmission cable 6. The same applies to the control signal, the synchronization signal, and the clock signal from the control device 9 to the camera head 5 through the first transmission cable 6.

The display device 7 is configured as a display using a liquid crystal or an organic electro luminescence (EL) and displays an image based on a video signal from the control device 9 under the control of the control device 9.

One end of the second transmission cable 8 is detachably connected to the display device 7 and the other end thereof is detachably connected to the control device 9. Then, the second transmission cable 8 transmits a video signal processed by the control device 9 to the display device 7.

The control device 9 corresponds to a medical image processing device according to this disclosure. The control device 9 includes a central processing unit (CPU) and the like and comprehensively controls the operations of the light source device 3, the camera head 5, and the display device 7.

Furthermore, a detailed configuration of the control device 9 will be described later.

One end of the third transmission cable 10 is detachably connected to the light source device 3 and the other end thereof is detachably connected to the control device 9. Then, the third transmission cable 10 transmits the control signal from the control device 9 to the light source device 3.

Configuration of Camera Head

Next, a configuration of the camera head 5 will be described.

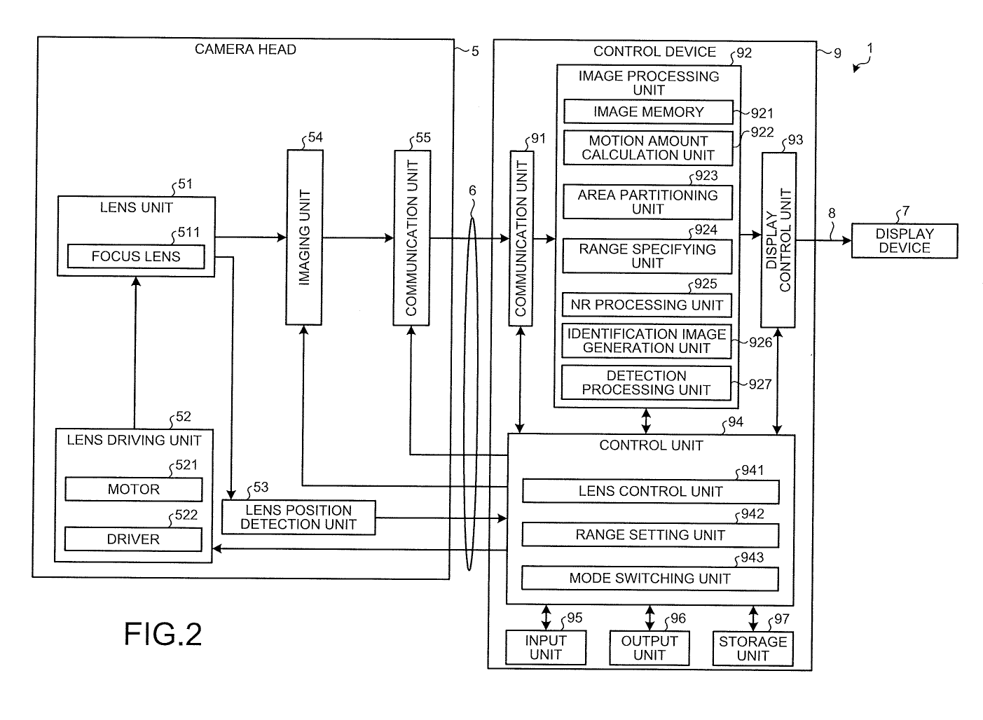

FIG. 2 is a block diagram illustrating the camera head 5 and the control device 9.

Incidentally, for convenience of description, FIG. 2 does not illustrate the connectors CN1 and CN2 provided to connect the first transmission cable 6 to the control device 9 and the camera head 5, respectively, and connectors provided to connect the second transmission cable 8 to the control device 9 and the display device 7, respectively.

The camera head 5 includes, as illustrated in FIG. 2, a lens unit 51, a lens driving unit 52, a lens position detection unit 53, an imaging unit 54, and a communication unit 55.

The lens unit 51 includes a plurality of lenses movable along an optical axis and forms the subject image condensed by the insertion unit 2 on an imaging surface of the imaging unit 54. At least one of the plurality lenses in the lens unit 51 is a focus lens 511, as illustrated in FIG. 2.

The focus lens 511 includes one or a plurality of lenses that is movable along an optical axis to adjust a focus.

Further, the lens unit 51 is provided with a focus mechanism (not illustrated) which moves the focus lens 511 along an optical axis.

Referring to FIG. 2, the lens driving unit 52 includes a motor 521 which operates the above-described focus mechanism, and a driver 522 which drives the motor 521. Then, the lens driving unit 52 adjusts the focus of the lens unit 51 under the control of the control device 9.

The lens position detection unit 53 includes a position sensor such as a photo interrupter and detects a lens position (hereinafter, referred to as a focus position) of the focus lens 511. Then, the lens position detection unit 53 outputs a detection signal corresponding to the focus position to the control device 9 through the first transmission cable 6.

The imaging unit 54 captures an image of the inside of the living body under the control of the control device 9. Although not illustrated in drawings specifically, the imaging unit 54 includes an imaging element such as a charge coupled device (CCD) or a complementary metal oxide semiconductor (CMOS) which receives the subject image condensed by the lens unit 51 and converts the subject image into an electric signal (analog signal) and a signal processing unit which performs a signal process on the electric signal (analog signal) from the imaging element and outputs an image signal (RAW signal (digital signal)).

The communication unit 55 functions as a transmitter which transmits the image signal (RAW signal (digital signal)) output from the imaging unit 54 through the first transmission cable 6 to the control device 9. The communication unit 55 is configured as, for example, a high-speed serial interface which is capable of transmitting an image signal to the control device 9 through the first transmission cable 6 at a transmission rate of 1 Gbps or more.

Configuration of Control Device

Next, a configuration of the control device 9 will be described with reference to FIG. 2.

As illustrated in FIG. 2, the control device 9 includes a communication unit 91, an image processing unit 92, a display control unit 93, a control unit 94, an input unit 95, an output unit 96, and a storage unit 97.

The communication unit 91 functions as a receiver which receives an image signal (RAW signal (digital signal)) output from the camera head 5 (the communication unit 55) through the first transmission cable 6. The communication unit 91 is configured as, for example, a high-speed serial interface which is capable of receiving an image signal from the communication unit 55 at a transmission rate of 1 Gbps or more.

The image processing unit 92 processes the image signal (RAW signal (digital signal)) which is output from the camera head 5 (the communication unit 55) and is received by the communication unit 91 under the control of the control unit 94. The image processing unit 92 includes an image memory 921, a motion amount calculation unit 922, an area partitioning unit 923, a range specifying unit 924, a noise reduction (NR) processing unit 925, an identification image generation unit 926, and a detection processing unit 927, as illustrated in FIG. 2.

The image memory 921 sequentially stores the image signal (RAW signal (digital signal)) which is output from the camera head 5 and is received by the communication unit 91 for each frame by a predetermined number of frames. That is, the image signal for a predetermined number of frames stored in the image memory 921 (the captured image for a predetermined number of frames) is sequentially rewritten to the captured image newly captured by the camera head 5.

The motion amount calculation unit 922 performs a motion amount calculation process of comparing the captured image (hereinafter, referred to as the current captured image) output from the camera head 5 and received by the communication unit 91 with the captured image (hereinafter, referred to as the previous captured image) stored in the image memory 921 and captured by the camera head 5 immediately temporally before the current captured image (before one frame) and calculating the motion amount from the previous captured image for each area of the current captured image (for each pixel in the first embodiment). Incidentally, the current captured image corresponds to a first image according to this disclosure. Additionally, the previous captured image corresponds to a second image according to the disclosure. Here, the second image according to the disclosure is not limited to the previous captured image and may be the captured image captured before several frames as long as the captured image is captured by the camera head 5 temporally before the current captured image.

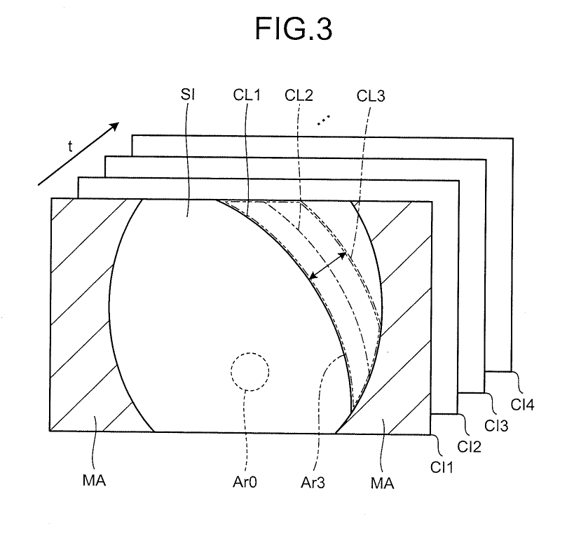

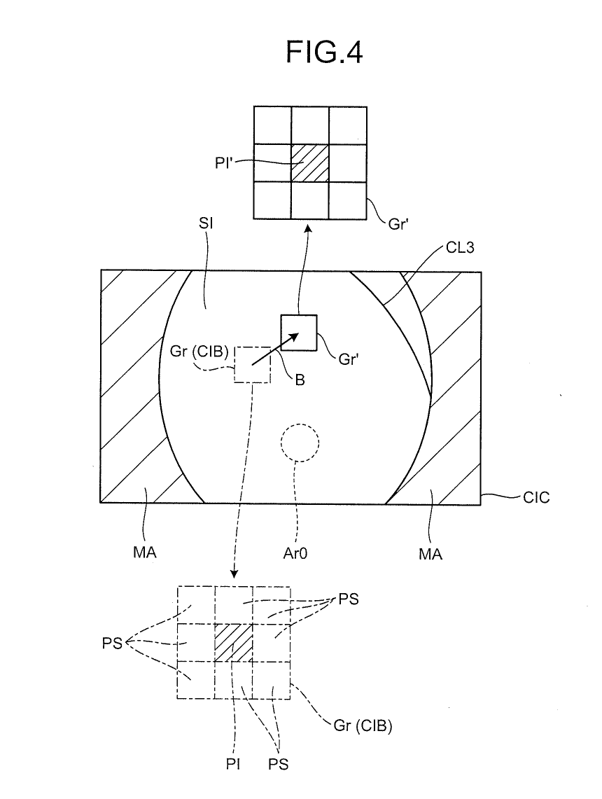

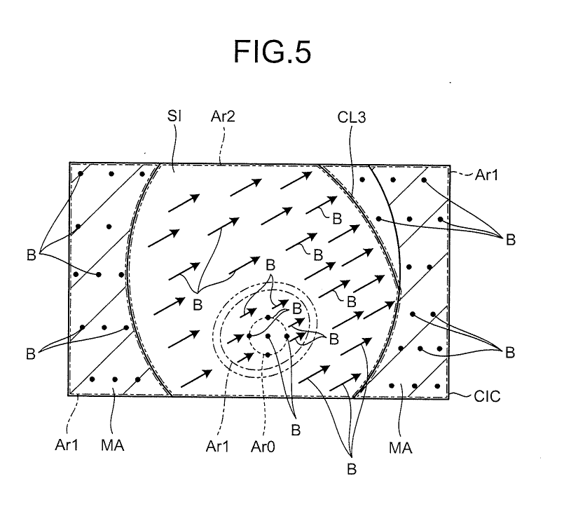

FIGS. 3 to 5 are diagrams illustrating an example of a motion amount calculation process. Specifically, FIG. 3 is a diagram in which captured images CI1 to CI4 captured by the camera head 5 are temporally arranged (in a direction indicated by an arrow t). Here, light (subject image) which is reflected from an inside of the living body and is condensed by the insertion unit 2 has a substantially circular cross-section. For this reason, a subject image SI in the captured images CI1 to CI4 is substantially circular as illustrated in FIG. 3. That is, the captured images CI1 to CI4 include the subject image SI and a mask area MA other than the subject image SI. Further, FIG. 3 illustrates a case in which an image of pulsatory subject such as a heart is captured. Then, in FIG. 3, the contour of the subject changing with pulsation is expressed by a curve CL1 indicated by a solid line and curves CL2 and CL3 indicated by a one-dotted chain line. Further, in FIG. 3, an area Ar0 indicated by a dashed line indicates a portion which is harder than the other sites due to a tumor or the like. Moreover, FIGS. 4 and 5 are diagrams corresponding to FIG. 3 and respectively illustrate a current captured image CIC in which the contour of the subject changes from the curve CL1 to the curve CL3 of FIG. 3 with pulsation.

For example, the motion amount calculation unit 922 performs a motion amount calculation process by using a block matching method as illustrated in FIGS. 4 and 5.

Specifically, the motion amount calculation unit 922 selects a pixel-of-interest PI (FIG. 4) among all pixels of the previous captured image CIB (FIG. 4). Further, the motion amount calculation unit 922 selects a pixel group Gr (FIG. 4) including the pixel-of-interest PI and a plurality of neighboring pixels PS (FIG. 4) located neighboring the pixel-of-interest PI. Incidentally, in the example of FIG. 4, the number of the neighboring pixels PS is set to eight (the number of pixels in the pixel group Gr is set to nine of 3×3 matrix), but the number is not limited to eight and may be others (for example, the number of the neighboring pixels PS is set to twenty four (the number of pixels in the pixel group Gr is set to twenty five of 5×5 matrix)).

Next, the motion amount calculation unit 922 specifies a corresponding pixel group Gr′ (FIG. 4) having the highest correlation with the pixel group Gr from the entire area of the current captured image CIC. Then, the motion amount calculation unit 922 calculates a vector from the pixel-of-interest PI located at the center of the pixel group Gr of the previous captured image CIB to a corresponding pixel-of-interest PI′ located at the center of the corresponding pixel group Gr′ of the current captured image CIC as a motion vector B (FIG. 4) of the corresponding pixel-of-interest PI′.

The motion amount calculation unit 922 calculates the motion vector B for each pixel (the corresponding pixel-of-interest PI′) of the current captured image CIC as illustrated in FIG. 5 by sequentially performing the above-described process with respect to the pixel-of-interest PI of all pixels in the previous captured image CIB. In FIG. 5, the directions (the motion directions) of the motion vectors B are indicated by corresponding arrows and the magnitudes (the motion amounts) of the motion vectors B are indicated by the lengths of the corresponding arrows. The motion vectors B indicated by dots indicate their motion amounts are zero.

Incidentally, the motion amount calculation process is not limited to the above-described block matching method and other methods (for example, a gradient method) may be used.

The area partitioning unit 923 performs an area partitioning process of partitioning the entire area of the current captured image CIC into an immovable area in which the motion amount calculated by the motion amount calculation unit 922 is within a specific range and a motion area in which the motion amount is outside the specific range by using the specific range set in the control unit 94. In the first embodiment, the area partitioning unit 923 uses a threshold value as the specific range. In the example illustrated in FIG. 5, the area partitioning unit 923 partitions the entire area of the current captured image CIC into an immovable area Ar1 in which the motion amount calculated by the motion amount calculation unit 922 is smaller than the threshold value and a motion area Ar2 in which the motion amount is equal to or larger than the threshold value by using the threshold value set in the control unit 94. Incidentally, the immovable area Ar1 corresponds to a first area according to the disclosure, and the motion area Ar2 corresponds to a second area according to the disclosure.

The range specifying unit 924 specifies a pulsation range (a range from the most contracted state to the most relaxed state) of the subject by referring to a plurality of captured images stored in the image memory 921 on the basis of the motion amount calculated for each captured image in the motion amount calculation unit 922. In the example illustrated in FIG. 3, the range specifying unit 924 specifies an area Ar3 from the position of the curve CL1 to the position of the curve CL3 as the pulsation range. Further, the range specifying unit 924 specifies the pulsation cycle on the basis of the specified pulsation range Ar3 and specifies a timing (hereinafter, referred to as a detection timing) corresponding to a middle of the pulsation range Ar3 of the subject (a middle between the most contracted state and the most relaxed state). In the example illustrated in FIG. 3, the range specifying unit 924 specifies a timing in which the contour of the subject is located at the position of the curve CL2 along with pulsation as the detection timing.

The NR processing unit 925 performs a noise reduction (NR) process of removing random noise of the current captured image CIC by applying a time filter to the immovable area Ar1 of the current captured image CIC and applying a space filter to the motion area Ar2.

The identification image generation unit 926 performs an identification image generation process of generating the immovable area Ar1 of the current captured image CIC subjected to the NR process and the identification image obtained by identifying the motion area Ar2.

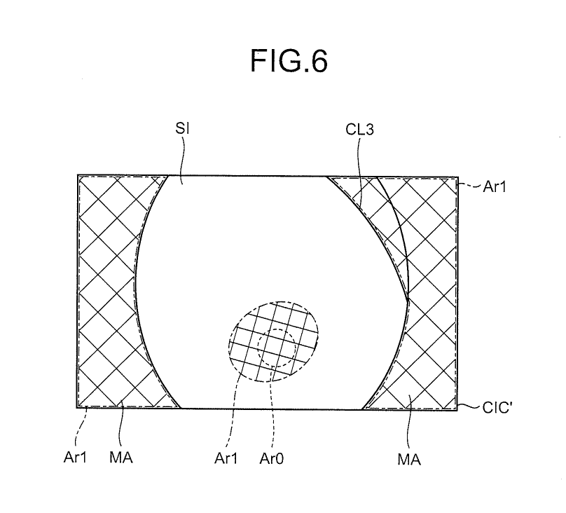

FIG. 6 is a diagram illustrating an identification image generation process. Specifically, FIG. 6 is a diagram corresponding to FIGS. 4 and 5 and illustrates an identification image CIC′ based on the current captured image CIC subjected to the NR process in a state where the contour of the subject changes from the position of the curve CL1 to the position of the curve CL3 of FIG. 3 along with pulsation.

In the example illustrated in FIG. 6, the identification image generation unit 926 generates the identification image CIC′ in which a specific color is given only to the immovable area Ar1 of the current captured image CIC after the NR process. Incidentally, although not illustrated in FIG. 6, the darkness or the brightness of the specific color given to the immovable area Ar1 becomes darker or brighter as the amount of motion calculated by the motion amount calculation unit 922 becomes larger. Additionally, the identification image generation unit 926 generates the identification image CIC′ in which a color different from that of the immovable area Ar1 is given to the pulsation range Ar3 of the current captured image CIC subjected to the NR process.

Furthermore, in FIG. 6, for convenience of description, a point in which a color different from that of the immovable area Ar1 is given to the pulsation range Ar3 is not illustrated.

The detection processing unit 927 performs a detection process to be illustrated below by using the current captured image CIC captured at the detection timing specified by the range specifying unit 924.

Specifically, the detection processing unit 927 performs a process of detecting a contrast or frequency component of the image in the detection area, detecting a maximum/minimum pixel or a luminance average value in the detection area by a filter or the like, determining a threshold value by comparison, and detecting a histogram on the basis of pixel information (for example, a luminance signal (Y signal)) for each pixel of a predetermined area (hereinafter, referred to as the detection area) including the pulsation range Ar3 in the entire current captured image CIC. Then, the detection processing unit 927 outputs the detection information (the contrast, the frequency component, the luminance average value, the maximum/minimum pixel, the histogram, and the like) obtained by the detection process to the control unit 94.

The image processing unit 92 outputs both images of the current captured image CIC subjected to the NR process and the identification image CIC′ based on the current captured image CIC to the display control unit 93.

The display control unit 93 generates a video signal for display on the basis of one of the identification image CIC′ and the current captured image CIC subjected to the NR process output from the image processing unit 92 under the control of the control unit 94. Then, the display control unit 93 outputs the video signal to the display device 7 through the second transmission cable 8.

The control unit 94 includes, for example, a CPU or the like and outputs a control signal through the first to third transmission cables 6, 8, and 10 to control the operations of the light source device 3, the camera head 5, and the display device 7 and to comprehensively control the operations of the control device 9. The control unit 94 includes, as illustrated in FIG. 2, a lens control unit 941, a range setting unit 942, and a mode switching unit 943.

The lens control unit 941 adjusts the focus of the lens unit 51 (changes the focus point) by operating the lens driving unit 52.

For example, the lens control unit 941 calculates a focus evaluation value for evaluating a focus state of the subject image SI included in the current captured image CIC on the basis of the detection information (the contrast or frequency component) output from the detection processing unit 927. Here, the lens control unit 941 sets the contrast detected by the detection processing unit 927 or the high frequency component of the frequency component detected by the detection processing unit 927 as the focus evaluation value. Incidentally, the focus evaluation value illustrates that a focus is matched as the value increases. Then, the lens control unit 941 performs an auto focus (AF) process of positioning the focus lens 511 to the focus position in which the subject image SI is in focus by a hill climbing method or the like on the basis of the focus position detected by the lens position detection unit 53 and the focus evaluation value.

The range setting unit 942 sets a specific range (a threshold value in the first embodiment) used in the area partitioning unit 923 on the basis of the operation signal from the input unit 95. Then, the range setting unit 942 outputs the set threshold value to the area partitioning unit 923.

The mode switching unit 943 switches observation modes between an immovable area observation mode where the identification image CIC′ is displayed on the display device 7 and a normal observation mode where the current captured image CIC subjected to the NR process is displayed on the display device 7 on the basis of the operation signal from the input unit 95. Then, the mode switching unit 943 outputs signals corresponding to the modes to the display control unit 93. That is, when receiving a signal corresponding to the immovable area observation mode from the control unit 94, the display control unit 93 generates a video signal for display on the basis of the identification image CIC′ based on the current captured image CIC subjected to the NR process, out of the current captured image CIC subjected to the NR process and the identification image CIC′, which have been output from the image processing unit 92, and outputs the generated video signal to the display device 7. On the other hand, when receiving a signal corresponding to the normal observation mode from the control unit 94, the display control unit 93 generates a video signal for display on the basis of the current captured image CIC subjected to the NR process, out of the current captured image CIC subjected to the NR process and the identification image CIC′ based on the current captured image CIC, which have been output from the image processing unit 92, and outputs the generated video signal to the display device 7. Incidentally, the immovable area observation mode corresponds to a first display mode according to this disclosure, and the normal observation mode corresponds to a second display mode according to the disclosure.

The input unit 95 includes an operation device such as a mouse, a keyboard, or a touch panel and receives a user's operation from a user such as a doctor. Then, the input unit 95 outputs an operation signal corresponding to the user's operation to the control unit 94. That is, the input unit 95 corresponds to an operation receiving unit according to the disclosure.

The output unit 96 includes a speaker or a printer and outputs various kinds of information.

The storage unit 97 stores a program executed by the control unit 94 or information necessary for the process of the control unit 94.

Operation Method of Medical Observation Apparatus

Next, an operation method of the medical observation apparatus 1 will be described.

Because detailed examples of the motion amount calculation process, the area partitioning process, and the identification image generation process have been already described, only an order of these processes and the image display will be described.

The image processing unit 92 sequentially performs the motion amount calculation process (Step S1: the motion amount calculation step), the area partitioning process (Step S2: the area partitioning step), and the identification image generation process (Step S3: the identification image generation step). Then, the image processing unit 92 outputs both images of the current captured image CIC subjected to the NR process and the identification image CIC′ based on the current captured image CIC to the display control unit 93.

After Step S3, the display control unit 93 determines whether a signal corresponding to the immovable area observation mode is input from the control unit 94 (Step S4).

When it is determined that the signal corresponding to the immovable area observation mode is input (Step S4: Yes), the display control unit 93 generates a video signal for display on the basis of the identification image CIC′ based on the current captured image CIC subjected to the NR process, out of the current captured image CIC subjected to the NR process and the identification image CIC′, which have been output from the image processing unit 92, and outputs the generated video signal to the display device 7. Accordingly, the display device 7 displays the identification image CIC′ (Step S5). Then, the control device 9 ends the operation method.

On the other hand, when it is determined that the signal corresponding to the normal observation mode is input (Step S4: No), the display control unit 93 generates a video signal for display on the basis of the current captured image CIC subjected to the NR process. out of the current captured image CIC subjected to the NR process and the identification image CIC′ based on the current captured image CIC subjected to the NR process, which have been output from the image processing unit 92, and outputs the generated video signal to the display device 7. Accordingly, the display device 7 displays the current captured image CIC subjected to the NR process (Step S6). Then, the control device 9 ends the operation method.

According to the above-described first embodiment, the following effect is obtained.

The control device 9 according to the first embodiment compares the current captured image CIC with the previous captured image CIB and calculates the motion amount from the previous captured image CIB for each pixel of the current captured image CIC. Further, the control device 9 partitions the entire area of the current captured image CIC into the immovable area Ar1 in which the motion amount is smaller than the threshold value and the motion area Ar2 in which the motion amount is equal to or larger than the threshold value. Furthermore, the control device 9 generates the identification image CIC′ that allows the immovable area Ar1 and the motion area Ar2, which are in the current captured image CIC, to be identified.

Here, because the site Ar0 such as a tumor is harder than the other sites (FIG. 6), the site does not easily move in comparison with the other portions. For this reason, the site Ar0 such as a tumor is included in the immovable area Ar1 of the identification image CIC′ as illustrated in FIG. 6.

Thus, a doctor or the like can easily find and diagnose a tumor or the like by checking the identification image CIC′ displayed on the display device 7. That is, the control device 9 according to the first embodiment may improve convenience.

Further, in the control device 9 according to the first embodiment, the threshold value used when performing the area partitioning process may be changed in response to a user's operation.

For this reason, because it is possible to narrow or widen the immovable area Ar1 by operating the input unit 95, a doctor or the like can more easily find and diagnose a tumor or the like.

Further, in the control device 9 according to the first embodiment, the identification image CIC′ in which a specific color may be given only to the immovable area Ar1 is generated. In particular, the darkness or brightness of the specific color given to the immovable area Ar1 is darkened or brightened as the motion amount increases.

For this reason, a doctor or the like can more easily find and diagnose a tumor or the like by checking the darkness or brightness of the specific color.

Further, in the control device 9 according to the first embodiment, the immovable area observation mode and the normal observation mode may be switched in response to a user's operation.

For this reason, when the identification image CIC′ does not need to be checked and the current observation image CIC needs to be checked, a doctor or the like can check the current observation image CIC by operating the input unit 95. Further, when the identification image CIC′ needs to be checked, a doctor or the like can check the identification image CIC′ by operating the input unit 95. Thus, it is possible to further improve convenience.

Further, in the control device 9 according to the first embodiment, the identification image CIC′ may be generated in which a color different from that of the immovable area Ar1 is given to the pulsation range Ar3.

For this reason, a doctor or the like can recognize an area (the pulsation range Ar3) with which a treatment tool or the like should not be in contact, by checking the identification image CIC′ displayed on the display device 7.

Further, in the control device 9 according to the first embodiment, a detection process may be performed by using the current captured image CIC captured at a detection timing corresponding to the middle of the pulsation range Ar3 of the subject.

For this reason, it is possible to reduce a focus shift between the most contracted state (see CL1 in FIG. 3) and the most relaxed state (see CL3 in FIG. 3) of the subject, which are caused by pulsatory movement of a subject, to a small focus shift between the most contracted state (or the most relaxed state) and the middle state (see CL2 in FIG. 3). Here, the small focus shifts from the middle state to the most contracted state and the most relaxed state are of the substantially same degree. For this reason, a doctor or the like can satisfactorily check the pulsation state of the subject from the current captured image CIC or the identification image CIC′ displayed on the display device 7.

### Second Embodiment

Next, a second embodiment will be described.

Hereinafter, the same reference numerals will be given to the same configurations as those of the above-described first embodiment and a detailed description thereof will be omitted or simplified.

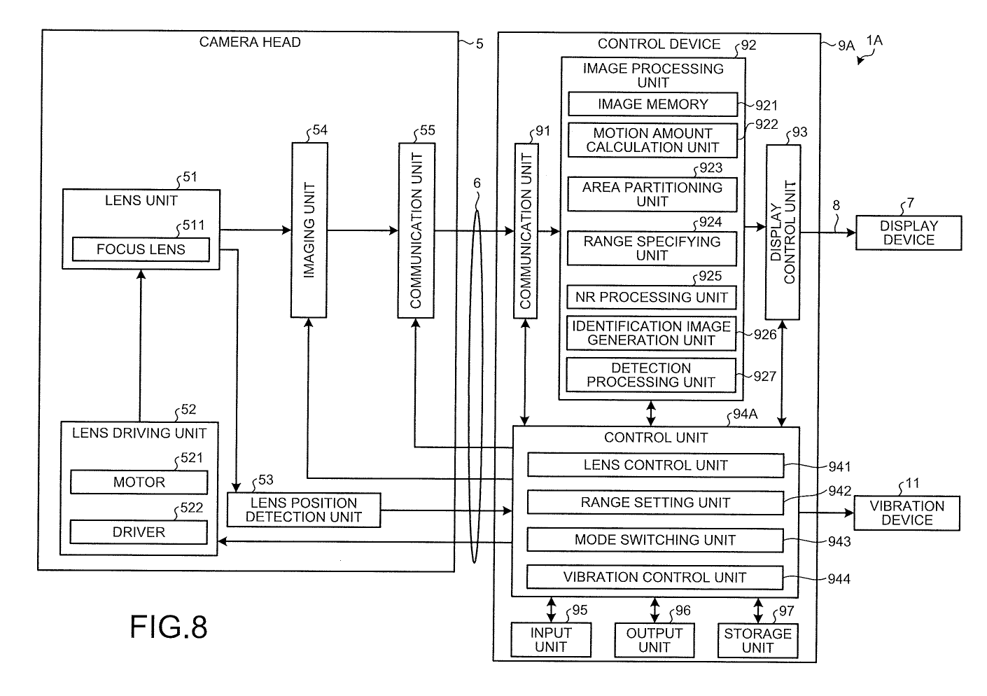

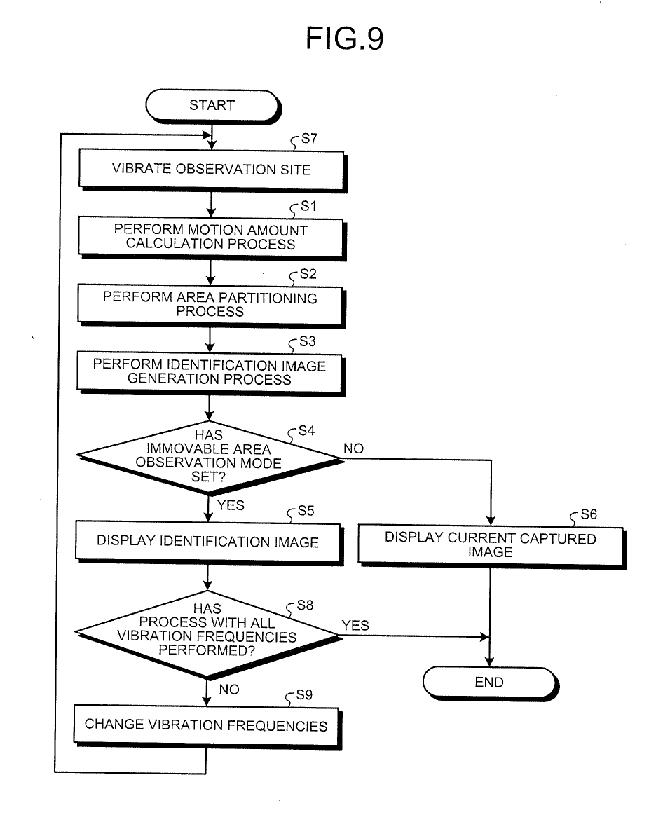

FIG. 8 is a block diagram illustrating a medical observation apparatus 1A according to the second embodiment. FIG. 9 is a flowchart illustrating an operation method of the medical observation apparatus 1A.

In the medical observation apparatus 1A according to the second embodiment, as illustrated in FIG. 8, a vibration device 11 is added to the medical observation apparatus 1 described in the above-described first embodiment.

The vibration device 11 includes a motor or a piezoelectric element. Then, the vibration device 11 is in contact with an outer surface of the subject or an observation site in the living body to apply a vibration to the outer surface or the observation site. Accordingly, the observation site is vibrated. Further, the vibration device 11 is configured to change the vibration frequencies (frequencies).

Further, in the medical observation apparatus 1A according to the second embodiment, a function of controlling the operation of the vibration device 11 is added to the control unit 94 in accordance with the addition of the vibration device 11.

Hereinafter, for convenience of description, a control device (a control unit) according to the second embodiment will be referred to as a control device 9A (a control unit 94A) and a function of controlling the operation of the vibration device 11 will be referred to as a vibration control unit 944. Here, the control device 9A corresponds to a medical image processing device according to this disclosure.

Hereinafter, a function of the vibration control unit 944 will be described with reference to FIG. 9.

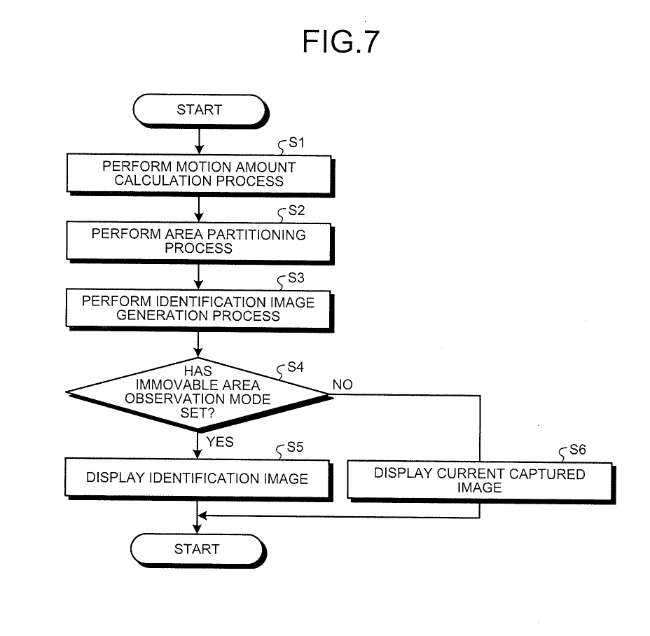

As illustrated in FIG. 9, an operation method of the medical observation apparatus 1A is different from the operation method of the medical observation apparatus 1 (FIG. 7) described in the above-described first embodiment in that Steps S7 to S9 are added. Hereinafter, only Steps S7 to S9 will be described.

Step S7 (the vibration step) is performed before Step S1.

Specifically, the vibration control unit 944 operates the vibration device 11 in Step S7. Accordingly, the observation site is vibrated. Then, the control device 9A proceeds a routine to Step S1.

Step S8 is performed after Step S5.

Specifically, in Step S8, the vibration control unit 944 determines whether the processes of Steps S1 to S5 are performed at all vibration frequencies changeable by the vibration device 11.

When it is determined that the processes are performed at all vibration frequencies (Step S8: Yes), the control device 9A ends the operation method.

Meanwhile, when it is determined that the processes are not performed at all vibration frequencies (Step S8: No), the vibration control unit 944 changes the vibration frequencies of the vibration device 11 (Step S9: the vibration frequency changing step). Subsequently, the control device 9A returns a routine to Step S7.

According to the above-described second embodiment, the following effects are obtained in addition to the same effects as those of the above-described first embodiment.

The medical observation apparatus 1A according to the second embodiment includes the vibration device 11. For this reason, it is possible to actively vibrate the observation site by the vibration device 11 even in the observation site of a non-pulsatory subject. That is, a doctor or the like can easily find and diagnose a tumor or the like included in the immovable area Ar1 by checking the identification image CIC′ displayed on the display device 7.

Further, in the medical observation apparatus 1A according to the second embodiment, the vibration device 11 can change the vibration frequencies.

Here, because the site Ar0 such as a tumor and the other sites have different hardness, natural vibration frequencies (resonance frequencies) are different. That is, for example, because the vibration frequencies are matched with the natural vibration frequencies of the other sites by adjusting the vibration frequencies of the vibration device 11, it is possible to set the site Ar0 such as a tumor in a non-vibration state while vibrating only the other sites. For this reason, a doctor or the like more easily finds and diagnoses a tumor or the like included in the immovable area Ar1 by checking the identification image CIC′ displayed on the display device 7.

### Third Embodiment

Next, a third embodiment will be described.

Hereinafter, the same reference numerals will be given to the same configurations as those of the above-described first embodiment and a detailed description thereof will be omitted or simplified.

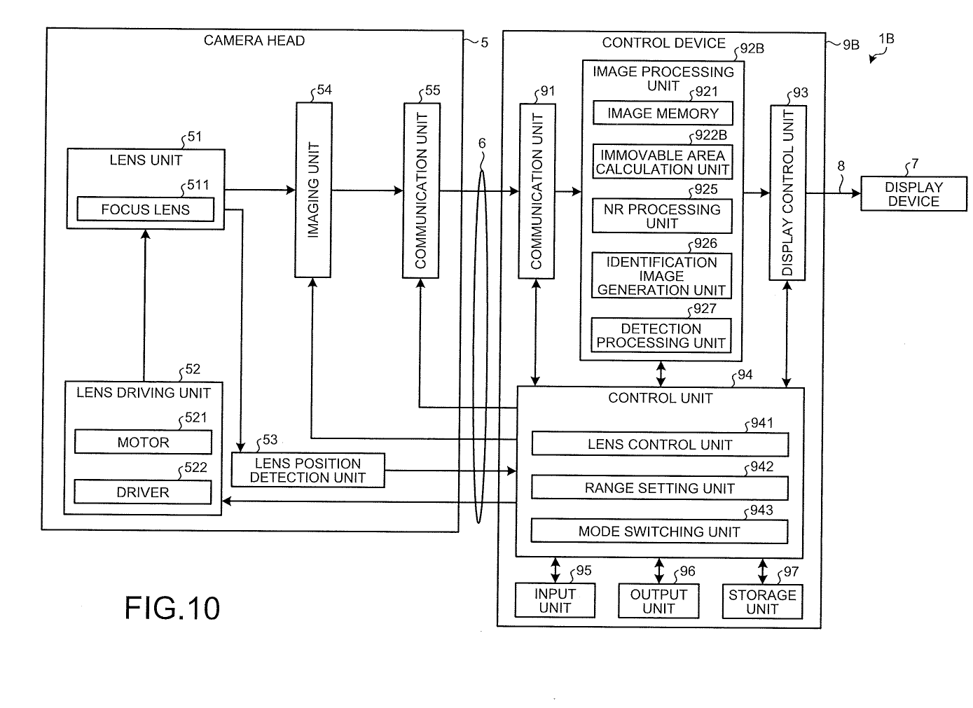

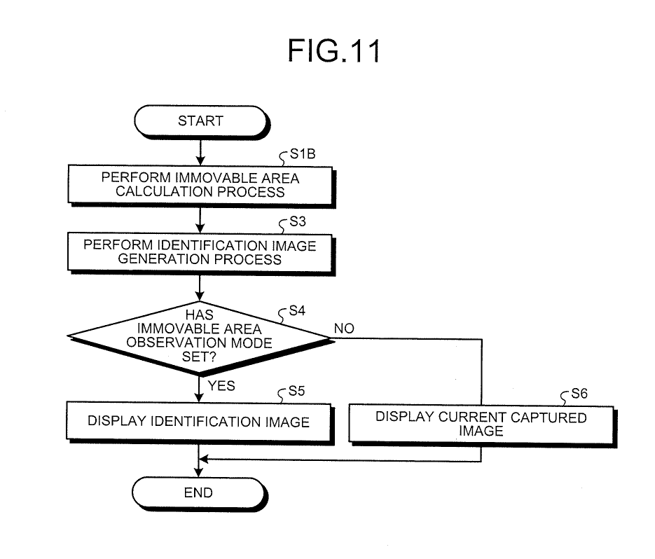

FIG. 10 is a block diagram illustrating a medical observation apparatus 1B according to the third embodiment. FIG. 11 is a flowchart illustrating an operation method of the medical observation apparatus 1B.

The medical observation apparatus 1B according to the third embodiment is different from the medical observation apparatus 1 described in the above-described first embodiment in that the function of specifying the immovable area and the motion area is different.

Hereinafter, for convenience of description, a control device (an image processing unit) according to the third embodiment will be referred to as a control device 9B (an image processing unit 92B). Furthermore, the control device 9B corresponds to a medical image processing device according to this disclosure. Then, the image processing unit 92B lacks for the area partitioning unit 923 and the range specifying unit 924 of the image processing unit 92B, which have been described in the above-described first embodiment. Additionally, the image processing unit 92B is provided with an immovable area calculation unit 922B instead of the motion amount calculation unit 922.

Incidentally, because the range specifying unit 924 is omitted in the detection processing unit 927 according to the third embodiment, a detection process is performed by using the current captured image CIC captured at a default timing or a timing corresponding to a user's operation to the input unit 95 or an input unit (not illustrated) provided in the camera head 5.

Hereinafter, a function of the immovable area calculation unit 922B will be described with reference to FIG. 11.

As illustrated in FIG. 11, an operation method of the medical observation apparatus 1B is different from the operation method of the medical observation apparatus 1 described in the above-described first embodiment (FIG. 7) in that Step S1B is adopted instead of Step S1 and Step S2 is omitted. Hereinafter, only Step S1B will be described.

In Step S1B (the immovable area calculation step), the immovable area calculation unit 922B compares the pixel values at the pixels corresponding to the current captured image CIC and the previous captured image CIB (at the pixels having the same pixel position) and calculates an area formed by the pixel positions having the same pixel value in the current captured image CIC as the immovable area. Further, the immovable area calculation unit 922B calculates an area other than the immovable area in the current captured image CIC as the motion area. Furthermore, similarly to the above-described first embodiment, an image compared with the current captured image CIC is not limited to the previous captured image CIB and may be the captured image captured before several frames as long as the captured image is captured by the camera head 5 temporally in advance with respect to the current captured image CIC. Then, the control device 9B proceeds a routine to Step S3.

Even when the immovable area and the motion area are calculated as in the above-described third embodiment, the same effect as that of the above-described first embodiment is obtained.

Modifications

Although a mode for carrying out the disclosure has been described so far, the disclosure is not limited only to the above-described first to third embodiments.

In the above-described first and second embodiments, the number of the threshold values used in the area partitioning process is not limited to one and may be two or more. Then, for example, when two threshold values are adopted, a range between the two threshold values may be a specific range according to the disclosure.

In the above-described first to third embodiments, the medical image processing device according to the disclosure is mounted on the medical observation apparatuses 1, 1A, and 1B each having a configuration in which the insertion unit 2 is a rigid endoscope, but the disclosure is not limited thereto. For example, the medical image processing device according to the disclosure may be mounted on the medical observation apparatus in which the insertion unit 2 is configured as a flexible endoscope. Further, the medical image processing device according to the disclosure may be mounted on a medical observation apparatus such as a surgical micro mirror (for example, see JP 2016-42981 A) which observes the inside of a subject (the inside of a living body) or a surface of a subject (a surface of a living body) of a predetermined viewing field area.

In the above-described first to third embodiments, the operation receiving unit according to the disclosure is provided in the control devices 9, 9A, and 9B, but the disclosure is not limited thereto. The operation receiving unit may be provided in the camera head 5.

In the above-described first to third embodiments, a configuration of a part of the camera head 5 or a configuration of a part of the control devices 9, 9A, and 9B may be provided in, for example, the connector CN1 or the connector CN2.

According to the medical image processing device, the medical observation apparatus, and the medical observation apparatus operation method of the disclosure, there is an effect that convenience can be improved.

Although the disclosure has been explained with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing device comprising:
a motion amount calculation unit which compares a first image captured of a subject with a second image captured of the subject, the second image having been captured temporally in advance with respect to the first image, and calculates a motion amount from the second image for each area in the first image;
an area partitioning unit which partitions the entire area of the first image into a first area in which the motion amount is within a predetermined range and a second area in which the motion amount is out of the predetermined range; and
an identification image generation unit which generates an identification image in which the first area and the second area of the first image are identified.

2. The medical image processing device according to claim 1, further comprising:
an operation receiving unit which receives a user's operation; and
a range setting unit which sets the predetermined range in response to the user's operation.

3. The medical image processing device according to claim 1,
wherein the identification image generation unit
generates the identification image in which a predetermined color is given only to one area of the first area and the second area of the first image.

4. The medical image processing device according to claim 3,
wherein the identification image generation unit
generates the identification image in which a darkness or brightness of the predetermined color is changed in accordance with the motion amount.

5. The medical image processing device according to claim 1, further comprising:
an operation receiving unit which receives a user's operation; and
a mode switching unit which switches between a first display mode of displaying the identification image on an external display device and a second display mode of displaying the first image on the display device in response to the user's operation.

6. A medical image processing device comprising:
an immovable area calculation unit which compares a first image captured of a subject with a second image captured of the subject, the second image having been captured temporally in advance with respect to the first image, at corresponding pixels, and calculates an immovable area in which no motion from the second image occurs in the first image; and
an identification image generation unit which generates an identification image in which the immovable area and the other areas of the first image are identified.

7. A medical observation apparatus comprising:
an imaging device which captures an image of a subject; and
the medical image processing device according to claim 1 which processes images captured temporally by the imaging device.

8. The medical observation apparatus according to claim 7, further comprising:
a vibration device which causes the subject to be vibrated.

9. The medical observation apparatus according to claim 8,
wherein vibration frequencies of the vibration device are changeable.

10. A medical observation apparatus comprising:
an imaging device which captures an image of a subject; and
the medical image processing device according to claim 6 which processes images captured temporally by the imaging device.

11. The medical observation apparatus according to claim 10, further comprising:
a vibration device which causes the subject to be vibrated.

12. The medical observation apparatus according to claim 11,
wherein vibration frequencies of the vibration device are changeable.
